(19) United States
(12) Plant Patent Application Publication
(10) Pub. No.: US 2003/0159188 P1
(43) Pub. Date: Aug. 21, 2003

(54) Apple tree named 'MC38'

(75) Inventor: Allan D. McLean, Harcourt (AU)

Correspondence Address: KLARQUIST SPARKMAN, LLP, One World Trade Center, Suite 1600, 121 S.W. Salmon Street, Portland, OR, 97204, US

(73) Assignee: A.D. McLean Investments Pty.

(21) Appl. No.: 10/081,731

(22) Filed: Feb. 21, 2002

(51) Int. Cl. A01H 5/00

(52) U.S. Cl. PLT/161

(57) ABSTRACT

A new apple variety distinguished by a spherical fruit shape, late fruit maturity, full-bodied flavor, and extensive striped overcolor.

The present invention relates to a new and distinct variety of apple tree named ‘MC38,’ which was discovered as a chance seedling growing among a uniform block of Pink Lady® ‘Cripps Pink’ variety (U.S. Pat. No. PP7880) apple trees in a cultivated area of Harcourt, Australia.

The ‘MC38’ variety is distinguished from other apple varieties due to the following unique combination of characteristics: the fruit is uniformly round, almost spherical, and more red in color than ‘Cripps Pink’; the fruit matures later than ‘Cripps Pink’ and Sundowner® ‘Cripps Red’ variety (patent pending); the fruit has a higher firmness rating than ‘Cripps Pink’.

Asexual reproduction of this new variety by grafting and budding onto rootstock shows that the foregoing and all other characteristics and distinctions come true to form and are established and transmitted through succeeding propagations.

The following detailed description concerns the original chance seedling, discovered in 1999, and progeny first asexually propagated in 1999. The original chance seedling and progeny have been observed growing in a cultivated area of Harcourt, Australia, and the first observed fruiting of the tree occurred in 1999.

Certain characteristics of this variety, such as growth and color, may change with changing environmental conditions (e.g., light, temperature, moisture), nutrient availability, or other factors. Color descriptions and other terminology are used in accordance with their ordinary dictionary descriptions, unless the context clearly indicates otherwise.

### BRIEF DESCRIPTION OF THE FIGUREs

FIG. 1 shows a branch of ‘MC38’ laden with fruit.

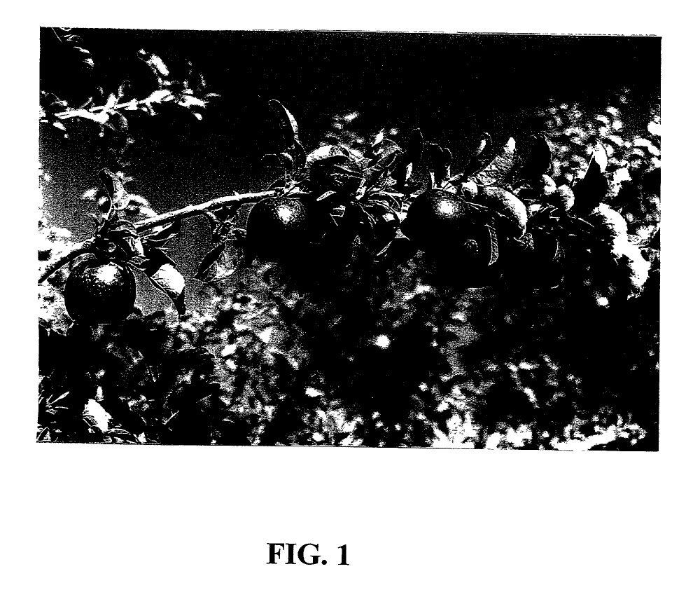

FIG. 2 is a close-up view of fruit, a branch, and leaves of ‘MC38.’

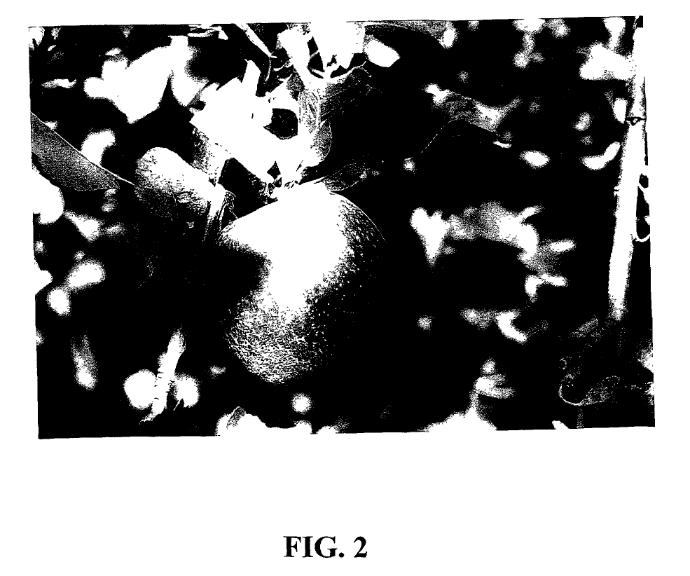

FIG. 3 is a comparison of fruit from ‘MC38’ and from ‘Cripps Pink.’

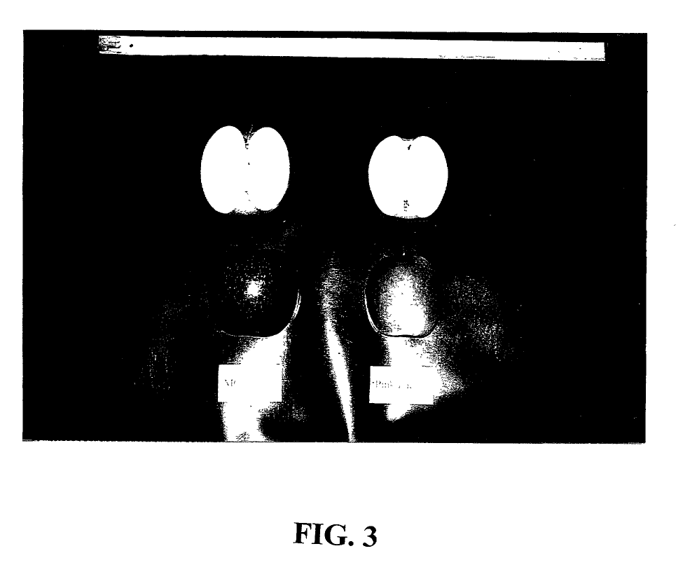

The colors of this illustration may vary with lighting conditions and, therefore, color characteristics of this new variety should be determined with reference to the observations described herein, rather than from these illustrations alone.

### DETAILED DESCRIPTION

The following detailed description of the ‘MC38’ variety is based on the originally identified tree and asexually reproduced progeny, growing in Harcourt, Australia.

Species: Malus domestica.

Parentage: Unknown. Seed parent believed to be ‘Cripps Pink.’

Tree: Observed trees were 2-3 years of age growing on MM-106 rootstock (unpatented) in Harcourt, Australia.

Vigor.—moderate.

Overall shape.—erect, but observed tree spreading through artificial means; young trees strongly apically dominant.

Trunk bark texture.—smooth.

Trunk bark color.—green-brown.

Patches or other markings.—prominent lenticel spotting.

Primary branches.—slender; branches emerge at an angle of about 70 to 80 degrees; exemplary two year old primary branches have been observed to have a caliper of about 14 to 15 mm.

Branch color.—one-year old branches are grey-green in color, while older branches are brown-green in color.

Branch pubescence.—absent.

Branch lenticels.—medium density, approximately 4 to 5 per square centimeter; oblong in shape; cream colored; and typically 1.0 to 1.5 mm long.

Internodes.—average internode length is about 20 to 35 cm for a two-year old shoot.

Bearing.—annual.

Hardiness.—comparable to ‘Cripps Red.’

Drought, disease and insect resistance.—comparable to ‘Cripps Red.’

Leaves:

Texture.—thin and leathery.

Sheen.—dull.

Length.—64.2 mm (average measurement from ten typical leaves).

Width.—44.3 mm (average measurement from ten typical leaves).

Petiole.—about 26.2 mm long and 1.3 mm in diameter; color is strong red at base fading to green at the top.

Margin.—serrate.

Tip shape.—acute.

Stipules.—About two stipules are present; very small and oppositely arranged; red in color; ovate shape with an obvate tip; less than 1 to 2 mm in length.

Leaf color.—Upper leaf surface: dark green. Lower leaf surface: light green. Vein: very light green.

Pubescence.—light pubescence on underside of leaf.

Flowers:

Size.—large.

Shape.—oval.

Color.—Flower — upper petal surface: pink. Flower — lower petal surface: pink.

Petals.—about five petals per flower.

Fruit (from unthinned trees):

Size.—medium to large, compared to ‘Cripps Pink’; average diameter of 72.7 mm for 10 typical ‘MC38’ apples.

Shape.—symmetrical and spherical; no ribbing; no lobes observed at calyx.

Cavity.—deep.

Basin.—medium wide; average width of 25.2 mm for 10 typical ‘MC38’ apples.

Pubescence.—none.

Stem.—short; average length of 19.5 mm and diameter of 1.92 mm for 10 typical ‘MC38’ apples; green in color with red stripes.

Locules.—about five in number; open; seed free from carpel wall.

Skin.—thick; glossy when polished; fruit does not get waxy in storage.

Lenticels.—low density, about 4 to 6 per square centimeter; conspicuous; cream-white in color.

Color.—General color effect: solid striped red overcolor that is very conspicuous; background color is yellow-green. Ground color: does not change greatly as fruit matures. Russetting: none present.

Ten typical apples of the ‘M38’ and of the ‘Cripps Pink’ cultivars from trees growing near to one another, were obtained on Apr. 7, 2000, and tested for certain properties. The averages of these properties were as follows:

| | ‘MC 38’ | ‘Pink Lady’ | ‘Cripps Red’ |
|---|---|---|---|
| Firmness | 9.2 | 8.8 | 8.9 |
| (pressure, kg/km²) | | | |
| Starch Index | 1.0 | 1.0 | 1.0 |
| On Scale of 1 (high starch) | | | |
| to 6 (low starch) | | | |
| Soluable Solids | 11.1 | 13.5 | 12.4 |
| (in percent) | | | |
| Red Color Rating | 4.5 | 0.7 | 0.7 |
| On a scale of 1 (low) | | | |
| to 5 (high) | | | |

A sample of 10 fruit showed all 10 having a 1.0 starch index.

Flavor.—tart and full-bodied, quantified at about 11.1 Brix.

Juiciness.—moderately juicy.

Flesh color.—white.

Aroma.—moderate aroma.

Core.—diameter at widest point is about 25.9 mm, compared to an overall fruit diameter of about 72.7 mm; shape is horizontal and oblong.

Seed.—about 0 to 2 seeds per cell; elongated in shape; seeds average about 7.82 mm long and 4.86 mm wide; brown in color.

Fruit production.—first picking date in the 2000 harvest in Harcourt, Australia, was about April 7, and last picking date was about April 28.

Storage.—fruit remains fresh at room temperature for 21 days, and can be stored up to four months in cold storage (34° F.).

Usage.—eating.

We claim:

1. A new and distinct variety of apple tree, substantially as herein shown and described.
